(19) United States
(12) Patent Application Publication
(10) Pub. No.: US 2007/0093897 A1
(43) Pub. Date: Apr. 26, 2007

(54) System and method for fusion cage implantation

(75) Inventors: Daniel E. Gerbec, Logan, UT (US); Joel Dever, Millville, UT (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Spine (in France), Cestas (FR)

(21) Appl. No.: 11/255,442

(22) Filed: Oct. 21, 2005

(51) Int. Cl. A61F 2/44; A61B 17/88; A61F 2/46

(52) U.S. Cl. 623/17.11; 600/431

(57) ABSTRACT

A system and method facilitate insertion of a fusion implant into the intervertebral space of a spine. The fusion implant may have a first bone engaging surface and a second bone engaging surface. An attachment interface on an insertion tool allows the implant to be releasably secured to the insertion tool, so that the insertion tool may be detached from the implant without requiring a threaded coupling. The implant may be positioned in two different orientations with respect to the insertion tool to permit usage of two different techniques to insert the implant into the intervertebral space. A recessed support member in the implant creates gaps between bone facing surfaces and the vertebral bodies such that bone graft material may occupy the gaps. The implant includes embedded radiographic markers which facilitate radiographic detection of the orientation of the implant through the surrounding tissue.

### BACKGROUND OF THE INVENTION

1.The Field of the Invention

The present invention relates generally to orthopedic devices, and, more specifically, to surgical devices and methods for fusing adjacent vertebrae.

2.The Relevant Technology

The spinal column is made up of thirty-three vertebrae separated by cushioning discs. Disease and trauma can damage these discs, creating instability that leads to loss of function and excruciating pain. Spinal fusion implants provide a successful surgical outcome by replacing the damaged disc and restoring the spacing between the vertebrae, eliminating the instability and removing the pressure on neurological elements that cause pain. The fusion is accomplished by providing an implant which recreates the natural intervertebral spacing and which has an internal cavity with outwardly extending openings. The internal cavity is commonly filled with osteogenic substances, such as autogenous bone graft or bone allograft, to cause the rapid growth of a bony column through the openings of the implant.

A variety of insertion tools exist for inserting fusion cage implants. Typically, the implantation tool is designed to fit a particular implant. Many implant tools currently in use require threading the implant on to the tool, inserting the implant, and then unscrewing the inserter to remove it from the patient. Cross-threading and/or stripping of threads may occur during this process, which can result in difficulty disengaging and removing the insertion tool. It would therefore be an improvement to provide a fusion implant insertion system that would include a system for releasably securing the implant to the insertion tool, so that disengaging the insertion tool from the implant would be simplified.

Fusion implants known in the art are held by their associated insertion tool in one position, requiring the use of one technique for insertion. Because some clinical situations require insertion of a fusion cage implant using a different approach, it would be desirable to be able to position the implant on the insertion tool in alternative positions. It would therefore be an improvement to provide a fusion implant insertion system in which the implant can be secured on the insertion tool in more than one configuration, so that an alternate technique for insertion may be employed for the same implant.

One challenge associated with spinal fusion cage implants is determining if the implant has been successfully positioned in the intervertebral space. Implants known in the art have markers which can be detected through tissue. However, correct alignment of the markers may be difficult to verify without checking the relative positioning of the markers from multiple viewpoints. It would therefore be an improvement to provide a fusion implant that is easier to check for proper alignment with the spinal column.

A key factor in successful spinal fusion via fusion cage implantation is the spreading and fusion of bone graft material through the implant. Known implants typically have openings to allow insertion of the bone graft material, and an interior space to hold the material. It would therefore be an improvement to provide a fusion implant that permits more comprehensive bone formation within the implant.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a perspective view illustrating a portion of the spine.

FIG. 2 is a perspective view of one embodiment of a fusion implant and an insertion tool.

FIG. 3 is an enlarged perspective view of the fusion implant shown in FIG. 2.

FIG. 4 is a cross sectional side view of the handle of the insertion tool shown in FIG. 2.

FIG. 5 is an enlarged cross sectional side view of the distal end of the insertion tool shown in FIG. 2 attached to the fusion implant shown in FIG. 2.

FIG. 6 is an enlarged cross sectional side view of the distal end of the insertion tool shown in FIG. 2 attached to the fusion implant shown in FIG. 2, showing an alternative placement of the fusion implant on the insertion tool.

FIG. 7 is an enlarged perspective view of the fusion implant shown in FIG. 2, showing the reverse side of the fusion implant from FIG. 3.

### DETAILED DESCRIPTION OF THE PREFERRED EMBODIMENTS

The present invention relates to orthopedic devices and related implantation instruments and methods. Although the examples provided herein relate to a fusion cage, the systems and methods described herein may be readily adapted for a wide variety of implants and procedures. Accordingly, the scope of the present invention is not intended to be limited by the examples discussed herein, but only by the appended claims.

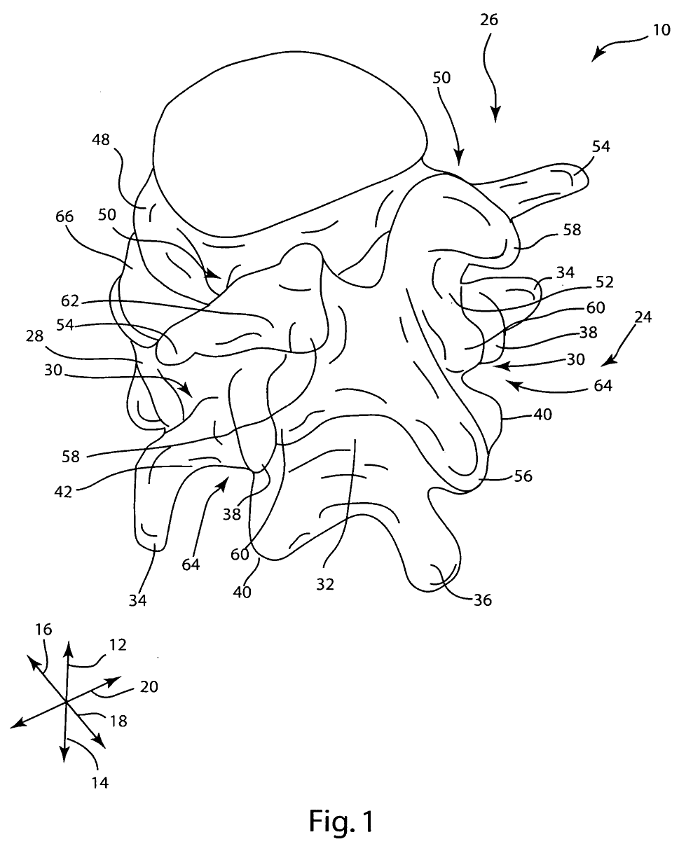

Referring to FIG. 1, a perspective view illustrates a portion of a spine 10. FIG. 1 illustrates only the bony structures; accordingly, ligaments, cartilage, and other soft tissues are omitted for clarity. The spine 10 has a cephalad direction 12, a caudal direction 14, an anterior direction 16, a posterior direction 18, and a medial/lateral axis 20, all of which are oriented as shown by the arrows bearing the same reference numerals. In this application, “left” and “right” are used with reference to a posterior view, i.e., a view from behind the spine 10. “Medial” refers to a position or orientation toward a sagittal plane (i.e., plane of symmetry that separates left and right sides from each other) of the spine 10, and “lateral” refers to a position or orientation relatively further from the sagittal plane.

As shown, the portion of the spine 10 illustrated in FIG. 1 includes a first vertebra 24, which may be the L5 (Fifth Lumbar) vertebra of a patient, and a second vertebra 26, which may be the L4 (Fourth Lumbar) vertebra of the patient. The systems and methods may be applicable to any vertebra or vertebrae of the spine 10 and/or the sacrum (not shown). In this application, the term “vertebra” may be broadly interpreted to include the sacrum.

As shown, the first vertebra 24 has a body 28 with a generally disc-like shape and two pedicles 30 that extend posteriorly from the body 28. A posterior arch, or lamina 32, extends between the posterior ends of the pedicles 30 to couple the pedicles 30 together. The first vertebra 24 also has a pair of transverse processes 34 that extend laterally from the pedicles 30 generally along the medial/lateral axis 20, and a spinous process 36 that extends from the lamina 32 along the posterior direction 18.

The first vertebra 24 also has a pair of superior facets 38, which are positioned toward the top of the first vertebra 24 and face generally medially. Additionally, the first vertebra 24 has inferior facets 40, which are positioned toward the bottom of the first vertebra 24 and face generally laterally. Each of the pedicles 30 of the first vertebra 24 has a saddle point 42, which is positioned generally at the center of the juncture of each superior facet 38 with the adjacent transverse process 34.

Similarly, the second vertebra 26 has a body 48 from which two pedicles 50 extend posteriorly. A posterior arch, or lamina 52, extends between the posterior ends of the pedicles 50 to couple the pedicles 50 together. The second vertebra 26 also has a pair of transverse processes 54, each of which extends from the corresponding pedicle 50 generally along the medial/lateral axis 20, and a spinous process 56 that extends from the lamina 52 along the posterior direction 18.

The second vertebra 26 also has a pair of superior facets 58, which are positioned toward the top of the second vertebra 26 and face generally inward. Additionally, the second vertebra 26 has inferior facets 60, which are positioned toward the bottom of the second vertebra 26 and face generally outward. Each of the pedicles 60 of the second vertebra 26 has a saddle point 62, which is positioned generally at the center of the juncture of each superior facet 58 with the adjacent transverse process 54.

The superior facets 38 of the first vertebra 24 articulate (i.e., slide and/or press) with the inferior facets 60 of the second vertebra 26 to limit relative motion between the first and second vertebrae 24, 26. Thus, the combination of each superior facet 38 with the adjacent inferior facet 60 provides a facet joint 64. The first and second vertebrae 24, 26 thus define two facet joints 64 that span the distance between the first and second vertebrae 24, 26. The inferior facets 40 of the first vertebra 40 and the superior facets 58 of the second vertebra 26 are part of other facet joints that control motion between the first and second vertebrae 24, 26 and adjacent vertebrae (not shown) and/or the sacrum (also not shown). The vertebrae 24, 26 are separated from each other by an intervertebral disc 66.

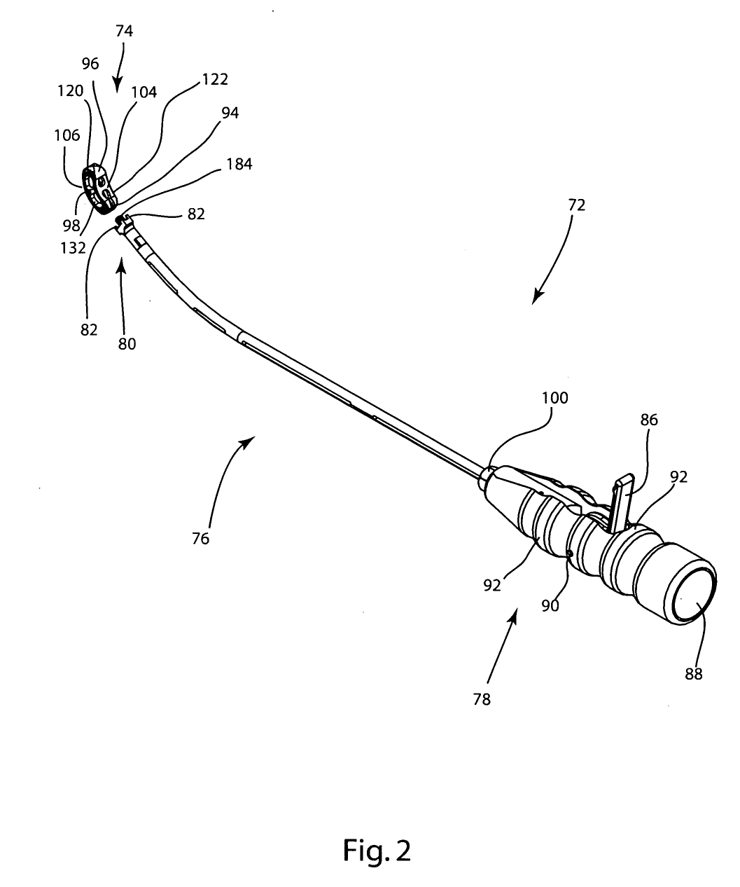

Referring to FIG. 2, a perspective view illustrates one embodiment of an implant 74, which may be termed a fusion cage, and an insertion tool 72. The implant 74 is designed for placement between bones and/or pieces of bone to facilitate fusing of the bone matter together. More precisely, the implant 74 of FIG. 2 is designed to be inserted between the vertebral bodies 28, 48 of the first and second vertebrae 24, 26, respectively, after removal of at least part of the intervertebral disc 66.

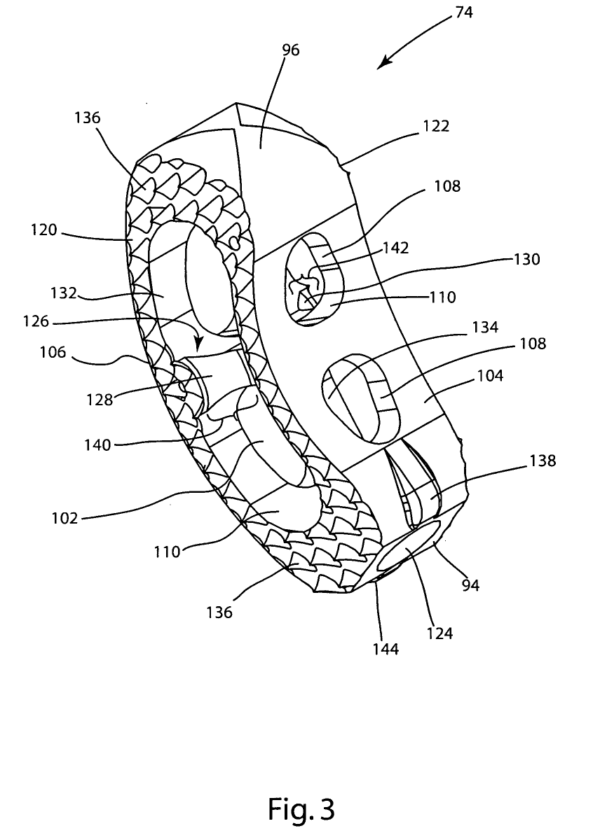

In the embodiment depicted in FIG. 2, the implant 74 has a generally arcuate shape with squared, box-like edges. The implant 74 has an outer wall 98 with a first bone engaging surface 120 and a second bone engaging surface 122, which extend between a first end 94 and a second end 96. Each of the bone engaging surfaces 120, 122 is shaped to abut one of the vertebral bodies 28, 48 of the vertebrae 24, 26, respectively. A first opening 132 on the first bone engaging surface 120 and a second opening 134 on the second bone engaging surface 122 communicate with a hollow interior space 102 encircled by the outer wall 98. A first support surface 104 and a second support surface 106 extend between the first end 94 and the second end 96. The outer wall 98 includes the first support surface 104 and the second support surface 106, which also extend between the edges of the first bone engaging surface 120 and the second bone engaging surface 122, thus forming a generally rectangular cross sectional shape. The first support surface 104 and second support surface 106 have a plurality of grafting ports which extend through implant 74 so as to communicate with the hollow interior space 102. The configuration of the implant 74 will be described in greater detail in connection with FIG. 3.

In the embodiment depicted in FIG. 2, the insertion tool 72 has a handle 78 at the proximal end and a stem 76 which terminates with an attachment interface 80 at the distal end. The handle 78 has a plurality of ergonomic grip rings 92 so as to make the handle 78 easy for the user to grip. As depicted in FIG. 2, the proximal end of the handle 78 terminates in a plug 88. A lever 86 is positioned on one side of the handle 78. A lever pin 90 forms an axis upon which the lever 86 can rotate. At the distal end of the handle 78, an adjustment sleeve 100 anchors the stem 76 to the handle 78.

As depicted in FIG. 2, the attachment interface 80 has a plurality of prongs 82 which encircle a collet 184. In this embodiment, the distal end of the stem 76 is slightly curved to facilitate the correct positioning of the implant 74 with respect to the vertebral bodies 28, 48 of the first and second vertebrae 24, 26, respectively. In other embodiments of this invention, the stem 76 may be straight for its entire length, or may be curved to provide a variety of configurations and overall angles.

Referring to FIG. 3, the implant 74 has a plurality of teeth 136 on the outer wall 98 of the first and second bone engaging surfaces, 120 and 122, respectively. The teeth 136 promote secure, substantially non-sliding abutment of the bone engaging surfaces 120, 122, with the vertebral bodies 28, 48, such that once implanted, the implant 74 substantially prevents relative motion between the first and second vertebral bodies 28, 48. The first bone engaging surface 120 of the outer wall 98 has a first opening 132 which communicates with the hollow interior space 102. Similarly, the second bone engaging surface 120 of the outer wall 98 has a second opening 134 which also communicates with the hollow interior space 102. In the embodiment depicted, the first and second openings 132, 134 comprise about 40 to 50 percent of the surface area of each of the first and second bone engaging surfaces 120, 122, respectively.

The outer wall 98 has an interior surface 110 that surrounds the hollow interior space 102. The interior surface 110 makes up the interior surfaces of the first support surface 104, the second support surface 106, the first end 94, and the second end 96. The interior surface 110 is bounded by the first and second openings 132, 134, a plurality of grafting ports 108, and an aperture 124 passing through the second end 96 of the implant 74. Within the hollow interior space 102, a support rib 126 extends from the interior surface 110, where it extends along the first support surface 104, to the interior surface 110, where it extends along the second support surface 106.

Thus, the support rib 126 spans the interior space 102. In this application, an element that “spans” a volume crosses the volume to leave space on either side of the element. The support rib 126 is only one of many possible supporting structures that may span the interior space 102 within the scope of the present invention. Other spanning members (not shown) may extend at different angles across the interior space 102 and/or between different locations on the outer wall 98. Such spanning members need not be integrated with the outer wall 98, but may instead be formed separately from the outer wall 98 and subsequently attached.

The support rib 126 has a first bone facing surface 128 and a second bone facing surface 130. The first bone facing surface 128 is recessed so as to form a first gap 140 between the first bone facing surface 128 and the vertebral body 28 or 48 to which it is adjacent after implantation. Similarly, the second bone facing surface 130 is recessed so as to form a second gap 142 between the second bone facing surface 130 and the vertebral body 28 or 48 to which it is adjacent after implantation. The first and second gaps 140, 142 allow space for occupation of bone graft material between the vertebral bodies 28, 48 and the bone facing surfaces 128,130. Accordingly, the first and second gaps 140, 142 permit the formation of a more complete bone column through the interior space 102, thereby more securely integrating the implant 74 with the vertebral bodies 28, 48.

As depicted in FIG. 3, an enlarged, perspective view illustrates the implant 74. The first support surface 104 and the second support surface 106 each include two grafting ports 108, which are positioned longitudinally along the midline of each support surface 104, 106. Each grafting port 108 communicates with the hollow interior space 102, facilitating spreading of bone graft material throughout the hollow interior space 102.

In the embodiment depicted in FIG. 3, the aperture 124 is a round opening located in on the first end 94. The aperture 124 is designed to fit around the collet 184 of the insertion tool 72, allowing the implant 74 to be releasably secured to the insertion tool 72. On the outer wall 98, proximate the first end 94, there is a first protrusion 138 which is located adjacent to the aperture 124, and extends toward the first support surface 104. The first protrusion 138 fits closely between the prongs 82 (as shown in FIG. 2) when the implant 84 is secured to the insertion tool 72. Similarly, on the opposite side of the aperture 124, the outer wall 98, proximate the first end 94, has a second protrusion 144. This second protrusion 144 extends from the aperture 124 toward the second support surface 106. When the implant 74 is secured to the insertion tool 72, the second protrusion 144 fits between the prongs 82 on the opposite side of the insertion tool 72. The two protrusions 138, 144 prevent the rotation of the implant 74 relative to the insertion tool 72 while the implant 74 is secured to the insertion tool 72.

The implant 72 is only one of many embodiments included within the scope of the invention. In other embodiments (not shown), implants need not have arcuate shapes, but may be cylindrical, rectangular, or otherwise differently shaped.

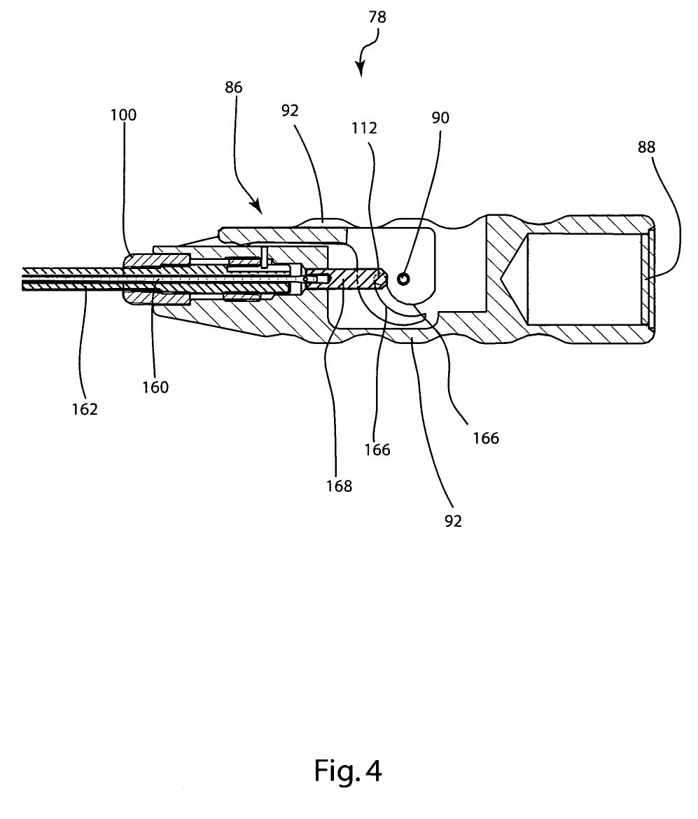

Referring to FIG. 4, a side elevation, section view illustrates the handle 78 of the insertion tool 72. As shown, the handle 78 houses a lever 86. Within the handle 78, the base of the lever 86 forms a curved cam surface 166. The cam surface 166 rotates on the axis of a lever pin 90 when the lever 86 is extended or retracted. A follower pin 112 is located within the curve of the cam surface 166. The proximal end of a follower 168 is attached to the follower pin 112. The distal end of the follower 168 attaches to a rod 160 which extends from the follower 168 out of the handle 78 to the distal end of the insertion tool 72. Surrounding the rod 160 is a hollow sleeve 162 that extends along the length of the stem 76. The adjustment sleeve 100 surrounds the proximal end of the hollow sleeve 162 to anchor the hollow sleeve 162 within the handle 78.

In the embodiment depicted in FIG. 4, when the lever 86 is extended away from the handle 78 such that it is generally perpendicular to the handle 78, the cam surface 166 rotates clockwise about the pin to slide on either side of the follower pin 112. As the cam surface 166 rotates, the follower 168 and the attached rod 160 are extended distally out of the handle 78. The hollow sleeve 162, which is anchored to the handle 78 by the adjustment sleeve 100, does not extend. When the lever 86 is retracted toward the handle 78, the cam surface 166 rotates back along the counterclockwise direction, and the follower 168 and the attached rod 160 are retracted proximally toward the handle 78. The rotation of the cam surface 166 may be terminated by contact with the follower pin 112, or by contact of the lever 86 with the adjoining stationary surfaces of the handle 78.

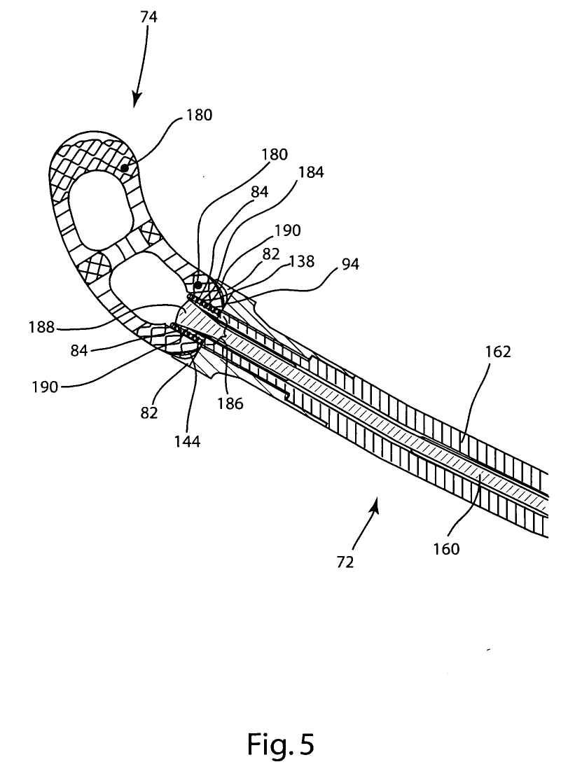

Referring to FIG. 5, a cross sectional side view of the releasable attachment of the implant 74 to the distal end of the insertion tool 72 is depicted. At the distal end of the insertion tool 72, the hollow sleeve 162 widens and terminates in two set of prongs 82. The prongs 82 are shaped so as to fit closely around the protrusions 138, 144 on the first end 94 of the implant 74. When viewed from a distal perspective, the four prongs form the corners of an approximate rectangle. In the center of the rectangle is a circular opening 186 at the end of the hollow sleeve 162.

A collet 184 is anchored within the circular opening 186 of the hollow sleeve 162. In the embodiment depicted, the collet 184 has four retention members 84 (only two of which are visible in FIG. 5) which are arranged in a circle. The edge of each retention member 84 is adjacent to the edge of the next retention member 84. The retention members 84 are each of an arcuate shape such that the four retention members 84 form a circle lining the circular opening 186, when viewed from a distal perspective. The retention members 84 extend distally out of the hollow sleeve 162, surrounded by the prongs 82. The outer facing surfaces of the retention members 184 are scored in a pattern of ridges, creating a ridged outer surface 190. Within the circle formed by the retention members 84, the rod 160 terminates in a bell-shaped end 188.

As depicted in FIGS. 2 and 5, the implant 74 may be releasably secured to the attachment interface 80 of the insertion tool 72. FIG. 2 depicts the implant 74 and the insertion tool 72 before attachment. During use, the lever 86 is extended from the handle 78 in the manner shown in FIG. 2, and the bell-shaped end 188 of the rod 160 extends out of the opening formed by the retention members 84. To releasably secure the implant 74, the aperture 124 in the first end 94 of the implant 74 is placed over the bell-shaped end 188 of the rod 160, and further over the four retention members 84. Next, the lever 86 is retracted toward the handle 78. This causes the rod 160 to be retracted proximally, along its axis, into the handle 78. As the rod 160 is retracted, the bell-shaped end 188 of the rod 160 contacts the retention members 84 and pushes them outward, expanding them apart from each other. As the retention members 84 expand, their ridged outer surfaces 190 engage the interior of the aperture 124 of the implant 74. As viewed in FIG. 5, when the lever 86 is fully retracted, the prongs 82 of the insertion tool 72 fit snugly around the protrusions 138, 144 of the implant, thus preventing rotation of the implant 74 relative to the insertion tool 72 while the implant 74 is attached to the insertion tool 72.

The implant 74 may then be inserted into the space between the vertebral bodies 28, 48 by, first, providing access to the space, and removing at least a portion of the intervertebral disc 66. Access may be provided from the posterior direction. The vertebrae 24, 26 may need to be distracted to temporarily widen the intervertebral space during insertion. Then, the surgeon may grasp and move the handle 78 to insert the implant 74 into the intervertebral space from an angle between the posterior direction 18 and the lateral direction 20. The surgeon may further manipulate the handle 78 to move the implant 74 to the proper orientation, so that the second support surface 106 is oriented toward the anterior direction 16. Such manipulation may involve striking the plug 88 with a hammer or the like to shift the implant 72 into the proper orientation between the vertebral bodies 28, 48.

Following implantation of the implant 74 between the vertebral bodies 28, 48 of the first and second vertebrae 24, 26, respectively, the lever 86 is again extended perpendicularly to the handle 78. Extending the lever 86 causes the follower 168 and the attached rod 160 to extend distally. As the rod 160 extends, the bell-shaped end 188 moves distally out of contact with the retention members 84, allowing the retention members 84 to contract. The ridged outer surfaces 190 of the retention members 84 disengage from the interior of the aperture 124 of the implant 74. Thus disengaged, the insertion tool 72 can be withdrawn from the patient, leaving the implant 74 in place.

The interaction of the collet 184 with the aperture 124 provides easy and secure engagement between the implant 74 and the insertion tool 72. Due to this secure engagement, impact against the plug 88 may be used to position the implant 74 with little fear that the implant 74 will accidentally become disengaged from the attachment interface 80. The engagement of the collet 184 with the aperture 124, also enables the insertion tool 72 to be easily disengaged from the implant 74.

The collet 184 and prongs 82 are only one example of an attachment interface according to the invention. According to other alternative embodiments (not shown), only two diametrically opposed retention members may be used. Such retention members may engage a round hole like the aperture 124, a flat-sided hole, a protrusion extending from some portion of the implant, or some other feature or combination of features. A movable retention feature may even be used in combination with a static retention feature to provide gripping action or outward retention force like that of the collet 184.

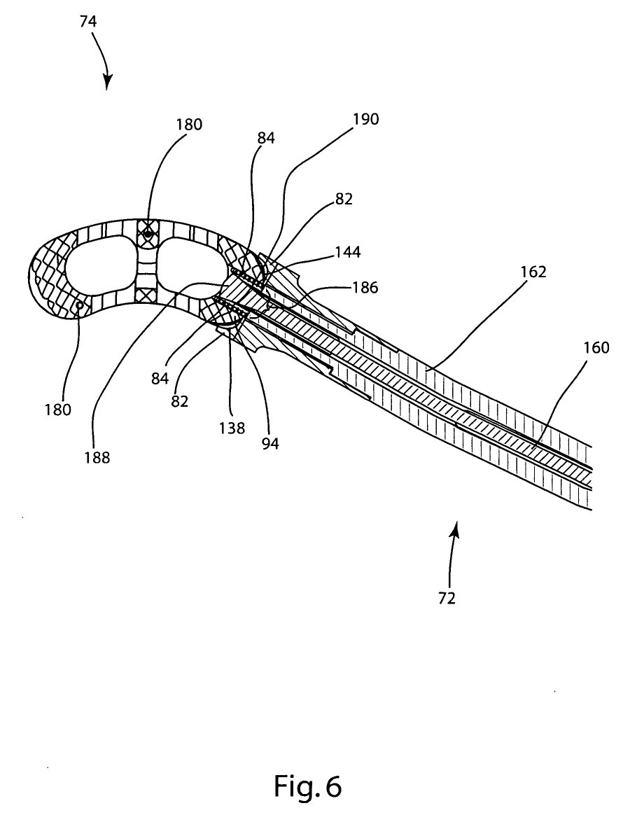

As shown in FIG. 6, the implant 74 may be releasably secured to the insertion tool 72 in an alternate configuration. In comparison to FIG. 5, in FIG. 6 the implant 74 has been turned on its longitudinal axis 180 degrees, so that the curve of the implant 74 is facing in the opposite direction. The protrusions 138, 144 on the first end 94 of implant 74 are shaped identically, so that each of the protrusions 138, 144 each can fit within either set of the prongs 82 on the distal end of the insertion tool 72. Positioning the implant 74 on the insertion tool 72 as shown in FIG. 5 permits usage of a first technique to insert the implant 74 into the intervertebral space. Positioning the implant 74 on the insertion tool 72 as shown in FIG. 6 permits usage of a second technique, different from the first technique, to insert the implant 74 into the intervertebral space.

The first and second techniques may differ by the manner in which access to the intervertebral space is obtained, by the angle at which the insertion tool 72 is held to place the implant 74, and/or a variety of other factors. The ability to use multiple techniques enable a surgeon to account for different morphologies of the spine and surrounding tissues, different implantation preferences, and other varying factors. The reversible engagement of the implant 74 on the insertion tool 72 enables the surgeon to select one of multiple insertion techniques without having to keep different implants or insertion tools on hand to accommodate them.

According to alternative embodiments (not shown), an implant may have more than two orientations with which it can be secured to the corresponding insertion tool. Such orientations may differ by any desirable angle. Indeed, a clocking feature having a multiplicity of engaging ridges and slots may be used to provide discrete, yet finely tunable control over the relative orientations of an implant and the corresponding insertion tool.

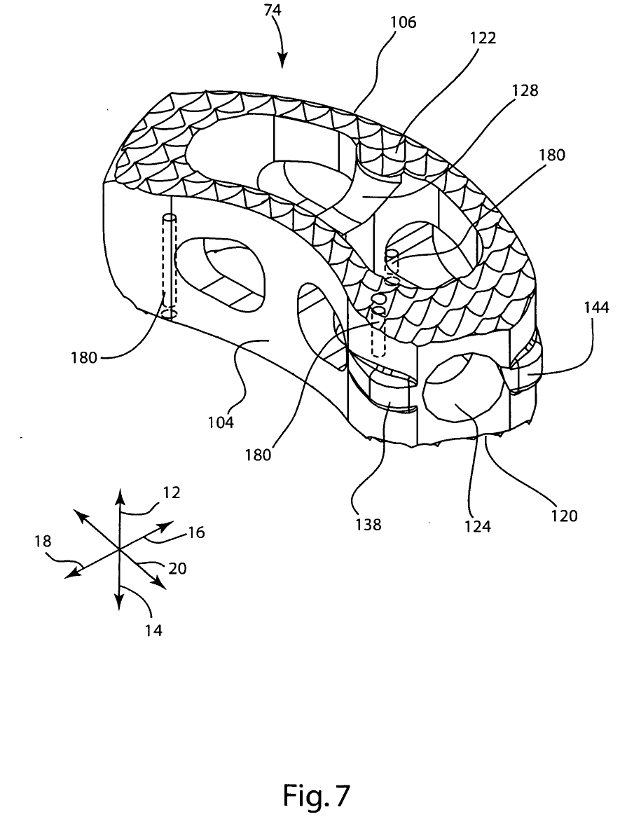

Referring to FIG. 7, three markers 180 are visible in the implant 74. In this embodiment, the markers 180 are composed of radiographic material, i.e., a material that is visible through tissue under radioscopy. A material such as tungsten may be used. Two of the markers 180 are embedded within the first bone engaging surface 120, and terminate so their ends are slightly recessed from the first bone engaging surface 120. A third marker 180 is similarly recessed in the second bone engaging surface 122. The markers 180 are positioned so that when the markers 180 are detected radiographically through tissue, the orientation of the implant 74 may be verified from a single viewpoint. Proper orientation of the implant 74 may be verified by detecting alignment of any two of the markers 180 with each other when viewed from one of the anterior direction 16, the posterior direction 18, the lateral direction 20, the cephalad direction 12, and the caudal direction 14.

For example, from the anterior or posterior directions 16, 18, the marker 180 proximate the second support surface 106 may appear to be equidistant between the markers 180 proximate the first support surface 104. From the cephalad and caudal directions 12, 14, the markers 180 proximate the first support surface 104 may appear to be aligned with each other along the same lateral axis of the patient. From the lateral direction 20, the markers 180 proximate the first support surface 104 may partially overlie each other, so that they can be distinguished from each other, yet their alignment indicates that they are on the same lateral axis of the patient.

In the alternative to the configuration of FIG. 7, a variety of different marker configurations may be used. Although the markers 180 are generally cylindrical, in alternative embodiments, they may have different shapes, and be distributed in the corresponding implant according to a variety of spacing configurations.

The present invention may be embodied in other specific forms without departing from its spirit or essential characteristics. It is appreciated that various features of the above-described examples can be mixed and matched to form a variety of other alternatives, each of which may have a different threading system according to the invention. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An orthopedic system comprising:
an implant shaped to be inserted into an intervertebral space of a spine, the implant comprising:
an outer wall defining a hollow interior space, the outer wall having a first bone engaging surface positioned to abut a first vertebral body adjacent to the intervertebral space; and
a support rib spanning the hollow interior space, the support rib comprising a first bone facing surface that is recessed with respect to the first bone engaging surface such that, after installation of the implant in the intervertebral space, a first gap exists between the first vertebral body and the first bone facing surface.

2. The orthopedic system of claim 1, wherein the outer wall further comprises a second bone engaging surface, wherein the outer wall is sized to enable the second bone engaging surface to abut a second vertebral body adjacent to the intervertebral space simultaneously with abutment of the first bone engaging surface against the first vertebral body, such that the implant substantially prevents relative motion between the first and second vertebral bodies.

3. The orthopedic system of claim 2, wherein the support rib further comprises a second bone facing surface that is recessed with respect to the second bone engaging surface such that, after installation of the implant in the intervertebral space, a second gap exists between the second vertebral body and the second bone facing surface.

4. The orthopedic system of claim 1, wherein the implant comprises a generally arcuate shape, when viewed from a cephalad viewpoint or a caudal viewpoint.

5. The orthopedic system of claim 1, further comprising an insertion tool releasably securable to the implant to facilitate positioning of the implant in the intervertebral space.

6. The orthopedic system of claim 5, wherein the outer wall further comprises an aperture, wherein the insertion tool comprises an attachment interface comprising a first retention member and a second retention member that are movable with respect to each other between a locked configuration, in which the first and second retention members press against opposing sides of the aperture, and a released configuration in which the first and second retention members are retracted from the opposing sides.

7. The orthopedic system of claim 5, wherein the insertion tool comprises an attachment interface releasably securable to an attachment interface of the implant in a first orientation of the implant with respect to the insertion tool to permit usage of a first technique to insert the implant into the intervertebral space, and in a second orientation of the implant with respect to the insertion tool to permit usage of a second technique, different from the first technique, to insert the implant into the intervertebral space.

8. The orthopedic system of claim 1, wherein the outer wall and the support rib are components of a body of the implant, the implant further comprising:
a first marker on the body; and
a second marker on the body;
wherein each of the first and second markers is detectable through tissue, wherein the first and second markers are relatively positioned such that, after installation of the implant in the intervertebral space at a proper orientation, the first and second markers are aligned with each other when viewed from one of an anterior viewpoint, a posterior viewpoint, a lateral viewpoint, a cephalad viewpoint, and a caudal viewpoint.

9. An orthopedic system comprising:
an implant shaped to be inserted into an intervertebral space of a spine, the implant comprising an aperture; and
an insertion tool comprising an attachment interface comprising a first retention member and a second retention member that are movable with respect to each other between a locked configuration, in which the first and second retention members press against opposing sides of the aperture, and a released configuration in which the first and second retention members are retracted from the opposing sides.

10. The orthopedic system of claim 9, wherein the implant comprises a first bone engaging surface and a second bone engaging surface, wherein the first and second bone engaging surfaces are positioned to abut first and second vertebral bodies adjacent to the intervertebral space to substantially prevent relative motion between the first and second vertebral bodies.

11. The orthopedic system of claim 9, wherein the implant comprises a generally arcuate shape, when viewed from a cephalad viewpoint or a caudal viewpoint.

12. The orthopedic system of claim 9, wherein the first and second retention members are components of an expandable collet of the attachment interface.

13. The orthopedic system of claim 12, wherein the attachment interface further comprises a rod comprising an axis, wherein the rod moves along the axis to trigger expansion of the expandable collet.

14. The orthopedic system of claim 13, wherein the rod comprises a tapered distal end extending through the collet such that the collet expands in response to retraction of the tapered distal end into the collet.

15. The orthopedic system of claim 9, wherein the insertion tool comprises a distal end comprising the attachment interface, and a proximal end comprising an actuation interface, wherein the attachment interface moves between the locked configuration and the released configuration in response to actuation of the actuation interface by a user.

16. The orthopedic system of claim 9, wherein the attachment interface is releasably securable to an attachment interface of the implant in a first orientation of the implant with respect to the insertion tool to permit usage of a first technique to insert the implant into the intervertebral space, and in a second orientation of the implant with respect to the insertion tool to permit usage of a second technique, different from the first technique, to insert the implant into the intervertebral space.

17. The orthopedic system of claim 9, wherein the implant comprises a body, the implant further comprising:
a first marker on the body; and
a second marker on the body;
wherein each of the first and second markers is detectable through tissue, wherein the first and second markers are relatively positioned such that, after installation of the implant in the intervertebral space at a proper orientation, the first and second markers are aligned with each other when viewed from one of an anterior viewpoint, a posterior viewpoint, a lateral viewpoint, a cephalad viewpoint, and a caudal viewpoint.

18. An orthopedic system comprising:
an implant shaped to be inserted into a body of a patient, the implant comprising an attachment interface; and
an insertion tool comprising an attachment interface releasably securable to the attachment interface of the implant in a first orientation of the implant with respect to the insertion tool to permit usage of a first technique to insert the implant into the body, and in a second orientation of the implant with respect to the insertion tool to permit usage of a second technique, different from the first technique, to insert the implant into the body.

19. The orthopedic system of claim 18, wherein the first and second orientations are separated by an angular displacement of 180°.

20. The orthopedic system of claim 18, wherein the implant comprises a generally arcuate shape, when viewed from a cephalad viewpoint or a caudal viewpoint.

21. The orthopedic system of claim 18, wherein the implant is shaped to be inserted into an intervertebral space of a spine of the body to substantially prevent relative motion of two vertebrae adjacent to the intervertebral space.

22. The orthopedic system of claim 21, wherein the first technique comprises insertion of the implant into the intervertebral space along a first posterior approach, wherein the second technique comprises insertion of the implant into the intervertebral space along a second posterior approach.

23. An orthopedic implant comprising:
a body shaped to be inserted into an intervertebral space of a spine;
a first marker on the body; and
a second marker on the body;
wherein each of the first and second markers is detectable through tissue, wherein the first and second markers are relatively positioned such that, after installation of the implant in the intervertebral space at a proper orientation, the first and second markers are aligned with each other when viewed from at least one of an anterior viewpoint, a posterior viewpoint, a lateral viewpoint, a cephalad viewpoint, and a caudal viewpoint.

24. The orthopedic implant of claim 23, wherein the body comprises a first bone engaging surface and a second bone engaging surface, wherein the first and second bone engaging surfaces are positioned to abut first and second vertebral bodies adjacent to the intervertebral space to substantially prevent relative motion between the first and second vertebral bodies.

25. The orthopedic implant of claim 23, wherein the first and second markers are radiographic.

26. The orthopedic implant of claim 25, wherein the first and second markers comprise metallic rods that are substantially radio-opaque.

27. The orthopedic implant of claim 23, wherein the first and second markers are relatively positioned such that, after installation of the implant in the intervertebral space at a proper orientation, the first and second markers are aligned with each other when viewed from a lateral viewpoint.

28. The orthopedic implant of claim 23, further comprising a third marker on the body, wherein the third marker is positioned to cooperate with the first and second markers to facilitate detection of whether the implant is at the proper orientation.

29. A method for implanting an implant in an intervertebral space of a spine, the method comprising:
inserting the implant into the intervertebral space such that a first bone engaging surface of an outer wall of the implant abuts a first vertebral body adjacent to the intervertebral space, wherein the outer wall defines a hollow interior space spanned by a support rib of the implant, the support rib comprising a first bone facing surface that is recessed with respect to the first bone engaging surface; and
inserting bone graft material into the hollow interior space such that the bone graft material occupies a first gap between the first vertebral body and the first bone facing surface.

30. The method of claim 29, wherein inserting the implant into the intervertebral space comprises abutting a second vertebral body adjacent to the intervertebral space with a second bone engaging surface of the outer wall, such that the implant substantially prevents relative motion between the first and second vertebral bodies.

31. The method of claim 30, wherein the support rib further comprises a second bone facing surface that is recessed with respect to the second bone engaging surface, the method further comprising inserting bone graft material into the hollow interior space such that the bone graft material occupies a second gap between the second vertebral body and the second bone facing surface.

32. The method of claim 29, further comprising releasably securing an insertion tool to the implant to facilitate positioning of the implant in the intervertebral space, wherein inserting the implant into the intervertebral space comprises actuating the insertion tool.

33. The method of claim 32, wherein releasably securing the insertion tool to the implant comprises:
inserting first and second retention members of the attachment interface of the insertion tool into an aperture of the outer wall; and
moving the attachment interface of the insertion tool from a released configuration, in which the first and second retention members are retracted from opposing sides of the aperture, to a locked configuration, in which the first and second retention members press against the opposing sides.

34. The method of claim 32, wherein releasably securing the insertion tool to the implant comprises releasably securing an attachment interface of the insertion tool to an attachment interface of the implant in one of a first orientation of the implant with respect to the insertion tool, and a second orientation of the implant with respect to the insertion tool, wherein inserting the implant into the intervertebral space comprises using one of a first technique to insert the implant in the first orientation, and a second technique different from the first technique to insert the implant in the second orientation.

35. The method of claim 29, wherein the outer wall and the support rib are components of a body of the implant, the implant further comprising a first marker on the body and a second marker on the body, the method further comprising:
positioning the implant at a proper orientation within the intervertebral space; and
detecting the first and second markers through tissue to verify positioning of the implant at the proper orientation by detecting alignment of the first and second markers with each other from one of an anterior viewpoint, a posterior viewpoint, a lateral viewpoint, a cephalad viewpoint, and a caudal viewpoint.

36. A method for implanting an implant in an intervertebral space of a spine, the method comprising:
inserting first and second retention members of an attachment interface of an insertion tool into an aperture of the implant;
moving the attachment interface of the insertion tool from a released configuration, in which the first and second retention members are retracted from opposing sides of the aperture, to a locked configuration, in which the first and second retention members press against the opposing sides; and
actuating the insertion tool to insert the implant into the intervertebral space.

37. The method of claim 36, wherein inserting the implant into the intervertebral space comprises abutting a first vertebral body adjacent to the intervertebral space with a first bone engaging surface of the implant, and abutting a second vertebral body adjacent to the intervertebral space with a second bone engaging surface of the implant to substantially prevent relative motion between the first and second vertebral bodies.

38. The method of claim 36, wherein the first and second retention members are components of an expandable collet of the attachment interface, wherein moving the attachment interface from the released configuration to the locked configuration comprises expanding the expandable collet.

39. The method of claim 38, wherein the attachment interface further comprises a rod comprising an axis, wherein expanding the expandable collet comprises moving the rod along the axis to trigger expansion of the expandable collet.

40. The method of claim 39, wherein the rod comprises a tapered distal end extending through the collet, wherein moving the rod along the axis comprises retracting the distal end into the collet.

41. The method of claim 36, wherein the insertion tool comprises a distal end comprising the attachment interface, and a proximal end comprising an actuation interface, the method further comprising actuating the actuation interface to trigger movement of the attachment interface from the released configuration to the locked configuration.

42. The method of claim 36, further comprising positioning the implant at one of a first orientation with respect to the insertion tool, and a second orientation with respect to the insertion tool, wherein inserting the implant into the intervertebral space comprises using one of a first technique to insert the implant in the first orientation, and a second technique different from the first technique to insert the implant in the second orientation.

43. The method of claim 36, wherein the implant comprises a body in which the aperture is formed, a first marker on the body, and a second marker on the body, the method further comprising:
positioning the implant at a proper orientation within the intervertebral space; and
detecting the first and second markers through tissue to verify positioning of the implant at the proper orientation by detecting alignment of the first and second markers with each other from one of an anterior viewpoint, a posterior viewpoint, a lateral viewpoint, a cephalad viewpoint, and a caudal viewpoint.
